(19) United States
(12) Patent Application Publication
(10) Pub. No.: US 2026/0215704 A1
(43) Pub. Date: Jul. 30, 2026

(54) MEDICAL DEVICE HAVING AN INDICATOR FIELD SENSITIVE TO AN ENVIRONMENTAL PARAMETER

(71) Applicant: Roche Diabetes Care GmbH, Mannheim (DE)

(72) Inventors: Alexander Steck, Hirschberg (DE); Kirill Sliozberg, Mannheim (DE)

(21) Appl. No.: 19/573,289

(22) Filed: Mar. 20, 2026

Related U.S. Application Data
(63) Continuation of application No. PCT/EP2024/075832, filed on Sep. 17, 2024.

(30) Foreign Application Priority Data
Sep. 20, 2023 (EP) 23 198 516.9

(51) Int. Cl. A61B 5/145

(52) U.S. Cl. CPC A61B 5/14532; A61B 2560/0242; A61B 2560/0271; A61B 2560/0276; A61B 2560/04

(57) ABSTRACT

A medical device includes a medical tool such as an inserter for a continuous glucose monitor. Typically a housing of the medical tool has an indicator area with an indicator field that irreversibly changes its appearance at a predetermined environmental parameter condition. In exemplary embodiments, the predetermined environmental parameter condition can be a predetermined humidity, a change in a predetermined humidity condition, a predetermined temperature or a predetermined temperature change. A controller is connected to the medical tool and is configured to determine the appearance of the indicator field at the time of use of the medical tool and is also configured to control the function of the medical tool based upon the determined appearance of the indicator field.

### RELATED APPLICATIONS

This application is a continuation of PCT/EP2024/075832, filed September 17, 2024, which claims priority to EP 23198 516.9, filed September 20, 2023, the entire disclosures of both of which are hereby incorporated herein by reference.

### BACKGROUND

This disclosure relates to a medical device comprising an indicator area, a control system for the function of a medical tool, a method of controlling the function of a medical tool, a use of a remote device to control the function of a medical tool as well as a use of a medical device or a control system.

It is known that medical devices, in particular those containing chemical and/or biochemical compounds, e.g., proteins, and/or synthetic substances, may be sensitive to environmental parameters, in particular humidity or humidity changes or temperature, i.e., may require handling and storage in a certain range of one or more of these environmental parameters and can be damaged when stored or handled outside of said range(s), in particular at high or low humidity, with great humidity changes and/or at high temperatures. A handling outside of the parameter range(s) may affect their ability to function properly, e.g., reduce or destroy their ability to function. In the case of a sensor using chemical and/or biochemical compounds for sensing an analyte, the calibration may be destroyed or the sensor may even be rendered unusable by handling outside of the acceptable range. This may, however, not be visible from the medical device, itself. Thus, a user may be harmed by relying on a medical device if during handling or storage the medical device had been exposed to environmental parameters outside of the acceptable range. Attempts to overcome this problem have been made. For example, EP 3 902 468 A1, U.S. Publication No. 2015/0246179 A1, JP 2021-528117 A1, EP 3787479 A2 and U.S. Publication No. 2022/0095968 A1 disclose ways of determining humidity or temperature exposure.

### SUMMARY

This disclosure teaches ways to reliably protect a user from potentially damaged medical devices, and the disclosure relates to the problem of reliably protecting a user from potentially damaged medical devices.

The medical device comprises a medical tool. The medical tool may in particular comprise one or more parts or components sensitive to an environmental parameter, in particular humidity or humidity changes. The medical tool may, for example, be or comprise a physiological sensor or a device for administering a drug. It may be a medical tool for insertion into living tissue, e.g., for subcutaneous insertion. The medical tool may in particular be an analyte sensor, in particular for sensing a particular analyte or concentration of an analyte, e.g., when inserted subcutaneously. The analyte may, e.g., be glucose or alcohol or a virus or a hormone or dopamine or ketones. The analyte may in particular be glucose. Alternatively, the medical tool may be a device for administering a drug, which may comprise a part or component sensitive to an environmental parameter, e.g., the drug, and/or another part sensitive to the environmental parameter. In particular, the drug may be a liquid drug, in particular insulin.

The medical device may, for example, comprise a housing in which parts of the medical tool and/or a controller as well as any optionally present other parts, e.g., batteries, may be arranged. The housing may have walls enclosing the inside of the housing (in which the parts of the medical tool and/or a controller and the optionally present other parts are arranged). In particular, the medical device may, e.g., be a continuous glucose monitoring system, in particular comprising a glucose sensor, or a continuous insulin delivery device, e.g., a continuous subcutaneous insulin infusion device. The medical device may comprise a patch for affixing the medical device to a user. The housing may, for example, be arranged on top of at least part of the patch of the medical device, i.e., on the side of the patch, which, in use, is opposite to the user’s skin. Thus, the patch may in particular also affix the housing to the user. The medical device may be delivered in an applicator that serves as help in applying the medical device to a user. The applicator may, in particular, be configured for inserting the medical tool into a tissue, in particular into the skin of a user, and may be removed after the medical device is applied for use.

The medical device comprises an indicator area connected to the medical tool. This connection may be direct, e.g., by the indicator area being arranged on the medical tool, or indirect, e.g., by indicator area being arranged on or comprised by a part of the medical device that is (directly or indirectly) connected to the medical tool. The indicator area may, for example, be arranged on the medical device, in particular on the medical tool or on a patch of the medical device or on the housing (in particular on the walls of the housing).

The indicator area may be arranged outside or inside of the housing. It may, in particular, be arranged on the outside of the housing (e.g., on a wall of the housing oriented toward the outside of the housing) or on the housing inside of the housing (e.g., on a wall of the housing oriented toward the inside of the housing). The connection between the indicator area and the medical tool is in particular carried out in a manner that makes sure the medical tool and indicator area experience the same environmental parameter during storage and handling.

For example, if the environmental parameter relates to humidity or a change of humidity, the medical tool and indicator area needs to be connected in a manner that the medical tool and indicator area experience the same humidity, e.g., they must be connected in manner allowing a free exchange of air. In particular, the medical tool and the indicator area may experience the same humidity or change of humidity if the indicator area is applied to the outside of the housing (in particular, if the medical device is a watertight medical device, and hence not gas-permeable).

As another example, if the environmental parameter relates to temperature, the medical tool and indicator are connected in a manner close to each other, thus ensuring that they experience the same temperature, but an exchange of air, although it may be present, may not be necessary. In particular, the medical tool and the indicator area may experience the same temperature or change of temperature, whether the indicator area is arranged outside or inside of the housing, in particular on the housing’s outside or on the housing’s inside. This is in particular even then the case if the housing or the medical device are watertight.

The indicator field or the entire indicator area comprising the indicator field, may be applied in the form of a sticker, of printing or a coating on a piece of the medical tool or a piece attached to it, e.g., the housing or a patch. The part sensitive to an environmental parameter is in the following also referred to as an indicator field. Examples for inks sensitive to an environmental parameter, e.g., humidity or temperature, are generally known. Examples of humidity sensitive inks that change their appearance when humidity changes are exemplarily disclosed in U.S. Publication No. 2018/0356379 A1.

The indicator area may, in addition to the indicator field, comprise an additional reference area (e.g., one or more additional reference field(s)), e.g., serving as reference for the original appearance and/or a changed appearance of the indicator field, as well as information about the device, e.g., in the form of a QR or bar code.

The indicator area may, in addition to a first indicator field, have a second and optionally further indicator fields, each of which may be sensitive to a further environmental parameter condition, e.g., humidity or temperature condition. These indicator fields may be arranged separate from each other and/or overlapping. With two, three or more indicator fields, additional information about storage parameters may be determinable from the indicator area, e.g., about a humidity condition and a temperature condition, or two humidity conditions, or two temperature conditions and optionally other environmental parameter conditions. Thus, it may, for example, be possible to determine from the appearance of a first indicator field whether an upper limit for humidity has been reached or exceeded during handling and storage and from a second indicator field whether a lower limit for humidity has been reached or fallen below. Exemplarily, in this text handling of one indicator field is described. However, corresponding steps as described for one indicator field may be carried out for each additional indicator field accordingly.

The medical device further comprises a controller connected to the medical tool, which is configured to control the function of the medical tool based upon the determined appearance of the indicator. The controller may also control the function of the medical tool based on other parameters and/or be configured to deliver information about the medical tool, e.g., measurement results or status indications of the medical tool, e.g., to a display or to a remote device.

In particular, the controller may be electrically connected and/or mechanically connected to the medical tool. The controller may have its own energy source, e.g., a small battery, in particular button cell, an antenna, and a communication interface for communicating with a remote device, e.g., by WiFi, Bluetooth, NFC or some other wireless communication protocol. Alternatively, the communication interface may communicate with the remote control in a wired fashion. The controller may also comprise a memory, e.g., an EEPROM, for storing information.

The controller may in particular be configured for starting the device’s function (activating the device), e.g., the measurement in the case of a sensor or the pumping of the drug in the case of a device for administering a drug. In particular, the medical device is configured that at the time of use of the medical tool, the appearance of the indicator field is determined. This may, for example, be done automatically, e.g., by the medical device itself, or be done by causing a user, e.g., via a remote device, e.g., a smartphone, or via some other way, to help in determining the appearance of the indicator field, e.g., by taking an image of the indicator field. Based on the appearance, the function of the device is then controlled. This may be together with an activation signal or upon an activation signal, which may, e.g., be received by the controller from a remote device or some other means, e.g., by pressing a button on the housing. Together with the activation signal or upon an activation signal, the controller may thus require information about the appearance of the indication field. In particular, the controller may be configured to not proceed with activating the device without a determination of the appearance of the indicator field having taken place.

In particular, the controller may be configured to control the function of the medical tool by disabling the medical tool based on the determined appearance of the indicator field. In particular, the device may disable the medical tool if the determined appearance of the indicator field indicates that the device was stored outside of the safe environment parameters. The medical tool may, for example, be disabled by blocking the activation signal or by disabling the medical tool in some other manner so that it cannot be used, e.g., by disabling the controller. The controller may additionally or alternatively be configured to control the function of the medical tool by adjusting a start parameter, e.g., the (measurement) scale, based on the determined appearance of the indicator field. In the following text, the (measurement) scale is used as example for a start parameter; however, this disclosure is not limited thereto. In particular for a factory calibrated system, environment parameters encountered during storage may change the calibration; the changes of the calibration may be known, e.g., from comparison measurements. Thus, if the indicator fields indicate the environmental parameters experienced during storage have changed the (measurement) scale of the medical tool, e.g., its sensitivity toward an analyte or the strength of a delivered drug, this (measurement) scale may be adjusted by the controller based on the determined appearance.

The controller may additionally or alternatively be configured to control the function of the medical tool by proceeding to a further step, e.g., activating the medical tool without modification if the indicator field indicates that the storage was within the acceptable environmental parameter range.

The indicator field is an environmental parameter sensitive part that is configured to (irreversibly) change its appearance at a predetermined environmental condition, e.g., a part that (irreversibly) changes its color at a predetermined environmental condition. For example, the environmental parameter sensitive part may be humidity sensitive or sensitive to a change of humidity (not the absolute value thereof), or sensitive to a temperature. Thus, the indicator field’s appearance may be different, when a predetermined environmental condition, e.g., humidity, is reached, exceeded or too low during transport or storage of the medical device, which is an advantage that protects a user from using a medical device which may be damaged when it is stored outside of certain environmental parameter conditions.

An environmental parameter may, for example, be humidity or temperature; in this text, humidity is often used as example for an environmental parameter. However, this disclosure also includes temperature or some other parameter to be the environmental parameter.

A predetermined environmental parameter condition, e.g., humidity condition, may in particular be that a) a predetermined environmental parameter, e.g., humidity, is reached or exceeded, b) a predetermined environmental parameter, e.g., humidity, is reached or fallen below, c) a predetermined environmental parameter, e.g., humidity, has been reached or exceeded for a certain amount of time, d) a predetermined environmental parameter, e.g., humidity, has been reached or fallen below for a certain amount of time, or e) the environmental parameter, e.g., humidity, has exceeded a predetermined environmental parameter, e.g., humidity, while at the same time a predetermined higher environmental parameter, e.g., humidity, is not reached, once or for a certain time.

The predetermined environmental parameter conditions, in particular the predetermined humidity conditions, usually depend on the medical device, in particular the medical tool. Typically, the predetermined environmental parameter condition is a threshold at which the properties of the medical tool change, e.g., render it less or more sensitive for the quantitative detection of an analyte in the case of a sensor, or render it less or more specific for the qualitative detection of an analyte in the case of a sensor, hence leading to an erroneous measurement of the determined analyte, or render it unusable or unreliable. These thresholds are typically known; for example, they can be determined in advance by testing the medical devices under certain known and determined environmental conditions, in particular humidity conditions. Thus, the change of appearance of the indicator field may be indicative of a change of a property of the medical device, in particular of the medical tool. In particular, the change in property can be a change of integrity of functioning and/or change of safety of the use of the medical device. Based on the described testing the predetermined environmental parameter conditions and storage time intervals can be determined that do or do not adversely affect the integrity of functioning and/or safety of the use of the medical device.

The environmental parameter sensitive part may be configured to change its appearance (from an original appearance) to a first appearance at a first predetermined environmental parameter condition and to change its appearance to a second appearance, that is in particular different from the first appearance, at a second predetermined environmental parameter condition, that is in particular different from the first predetermined environmental parameter condition. At the first predetermined environmental parameter condition, the medical tool may still be able to function, but a different start parameter, e.g., an adjustment of a (measurement) scale and/or calibration, may be necessary. Such a start parameter may in particular be or comprise that the device can still start, but the controller provides the measurement data in a manner such that user is warned of lower measurement precision and/or the device operation time may be limited, in particular by the controller, to a particular duration, as a medical device, in particular medical tool, that has been stored at improper environmental conditions may show signal drift earlier than one that has been properly stored.

The medical device may be configured such that at the time of use of the medical tool, the appearance of the indicator field is determined by a remote device, and a corresponding indication is provided to the controller.

The remote device may, for example, be a handheld remote device, e.g., a smartphone or tablet.

The remote device may have program instructions, e.g., an app, allowing use of the remote device with the medical device. The remote device may in particular be configured to communicate with the controller. It may be configured to send an activation signal and/or to disable the device and/or set a start parameter for the medical device, e.g., adjust the (measurement) scale. This may be done by sending an explicit signal or information, from which the controller can deduct the information.

The remote device may comprise a display or other interface for communicating with a user and program instructions, e.g., an app, configured to indicate to a user that a determination of the appearance of the indicator field is required before the medical device can be used. The remote device may in particular guide the user with regard to the steps necessary to be carried out to determine the appearance of the indicator field.

The remote device may further comprise hardware, e.g., a camera or other interface, for determining information about the appearance of the indicator field. This information may, e.g., be determined by a user providing information or by taking a picture or determining the appearance in another manner.

The appearance of the indicator field may be determined by reviewing its image. This may, for example, be done by a user looking at the field, optionally comparing it to a reference, e.g., a reference image or a reference area of the indicator area, and indicating the corresponding information, e.g., through inserting the information into a remote device or the medical device itself. A determination by reviewing its image may alternatively be carried out by taking an image through hardware, e.g., a camera or sensor, and using the corresponding image in order to determine the appearance of the indicator field, e.g., by (automatic) comparison to a reference image or reference area (reference field) of the indicator area, e.g., by automatic image processing.

Alternatively or additionally, the appearance of indicator field may be determined by electronic measurement. For example, the appearance of the indicator field may be determined by a reflectometer comprising a photodiode, or other sensor, e.g., camera. In particular, the reflectometer may comprise a light source, e.g., an LED to provide the illumination for the reflectometer, and a sensor, e.g., a photodiode, to measure reflection, in particular reflection intensity or reflection spectrum. The light source of the reflectometer as well as its sensor are in particular arranged such that the reflection of the indicator field is measured.

Such a reflectometer or other sensor readings may be calibrated to the appearance of the indicator field at fabrication time (corresponding to an unchanged appearance as would be displayed if the proper storage or handling conditions were present). The reflectometer or other sensor readings may in particular be calibrated to the reflection intensity at fabrication time, e.g., by running the reflectometer once at fabrication time and storing the measured sensor readings in the controller’s memory. Deviations from the reflectometer or other sensor readings from the calibrated value at a later time, in particular at activation time, are indicative of a change of appearance of the indicator field, and in particular thus of improper storage or handling conditions.

Alternatively, the reflectometer or other sensor readings may be compared or calibrated to measurement of a reference field the appearance of which is determined when the appearance of the indicator field is determined (in particular at the time of use of the medical tool, e.g., directly before, while or directly after the appearance of the indicator field is determined). Such a comparison or calibration may be particularly useful if there is stray light from the outside because it helps in avoiding falsification of the determined appearance of the indicator field by stray light.

In particular, the appearance of the indicator field may be determined by the medical device, in particular a measurement device attached to the controller.

For example, the medical device may comprise a reflectometer or other sensor to measure the light reflected from the indicator field, which may in particular be calibrated as explained above. The medical device, in particular the reflectometer, may comprise a light source, e.g., LED, to illuminate the indicator field, hence allowing measurement of the reflectance thereof.

The indicator area may be arranged on a transparent part of the housing, in particular on the outside of the housing. This way, it is exposed to the same environmental parameters as the medical tool. Transparent in this context may in particular mean transparent in one or more wavelength ranges used in determining the appearance of the indicator field, e.g., one or more or all the visible wavelength range(s). Transparent may mean that it has a transmittance of more than 50%, in particular of more than 90% for each wavelength of these wavelength range(s). In other wavelength ranges (not used in determining the appearance of the indicator field), the (transparent) part of the housing may be, but need not be transparent.

Alternatively or additionally, the indicator area may be arranged inside of a housing, in particular on the inner wall of a housing or arranged of some other part of the medical device in the housing. For example, if the environmental parameter is a temperature or a temperature change, the indicator area may be arranged on the inside or the outside of a housing, because the indicator area experiences the same temperature or temperature change as the medical tool on either side of the housing, even for a medical device having a watertight housing.

For an indicator area arranged inside of a housing, the housing may in particular be opaque, e.g., not let light from outside enter the housing (as there is no need for light to travel through the wall of the housing). Opaque may in particular mean that in the one or more wavelength ranges used in determining the appearance of the indicator field, e.g., one or more or all the visible wavelength range(s), transmittance is less than 5%, in particular less than 1%. In other wavelength ranges (not used in determining the appearance of the indicator field), the housing may be, but need not be, opaque.

Such an opaque housing may in particular, optionally together with other parts of the medical device, e.g., a patch, or with a part of a packaging of the medical device, form a closed opaque system into which no light from the outside can enter. Thus, the indicator area may, in this particular example, comprise only a single indicator field (for each environmental parameter condition) or consist of a single indicator field (for each environmental parameter condition) as the closed opaque system does not receive stray light from outside. Thus, in particular no reference field(s) may be necessary.

A reflectometer including a light source, e.g., LED, and a sensor for measuring the reflection, e.g., a photodiode, may be arranged within the housing, e.g., on a printed circuit board assembly.

The medical device may be configured such that the reflectance of the indicator field may be determined by the reflectometer comprised by the medical device.

As the medical device may comprise the medical tool and the housing, which may in particular be wrapped in the same cover, the environmental parameter conditions experienced by the medical tool and the housing during storage and handling may be the same. Thus, if the controller receives an activation signal, it may determine the appearance of the indicator field by measurement of the reflection thereof. If the measured value corresponds to the calibrated value, the controller needs not take any steps to disable the device. If the measured value deviates from the calibrated value, the controller may, depending on the determined appearance, adjust the (measurement) scale of the medical device or disable the medical device.

The medical device may be configured in such a manner that a signal is sent to a remote device indicating the result of the control of the function of the medical tool. This signal may in particular be sent by the controller and may be sent to a remote device, e.g., a smartphone. The remote device may be configured to display information based on the signal, e.g., in an app or on the general display, or to provide information based on the signal, e.g., a warning, e.g., by flashing a light or sounding an alarm, or providing the information to the user in some other manner. Alternatively or additionally, the result of the control of the function of the medical tool may be indicated in some other way, e.g., by an indication device, e.g., lamp or display or sound, attached to the medical device, e.g., the housing. In this manner, the user can be alerted if the medical tool is disabled or its (measurement) scale has been adjusted, e.g., because of decreased sensitivity or decreased lifetime of the medical tool.

The indicator field may be sensitive to humidity or humidity changes and/or to temperature or temperature changes.

The medical device may additionally comprise an activation label comprising an activation code required for activating the medical device, wherein the controller is configured to receive an activation signal from a remote device, and wherein the activation label comprises a temperature sensitive part that is configured to change its appearance at a predetermined temperature condition, in particular such that it masks at least part of the activation code if the predetermined temperature condition is reached.

The activation label and controller as well as the remote device may in particular be configured as further detailed in the European Patent Application titled “Medical Device comprising a temperature sensitive activation label” filed on June 9, 2023, with the European Patent Office as EP 23178 437.2, the entire disclosure of which is hereby incorporated herein by reference.

This disclosure further comprises a control system for the function of a medical tool comprising a medical device as described above and a remote device, e.g., a smartphone, as described above. In particular, the remote device may have means to communicate with the controller of the medical device, and hardware, e.g., a camera or interface, to receive information to determine the appearance of the indicator field. The remote device may in addition be configured for communicating with the controller of the medical device, in particular to send an activation signal and/or control the functioning of the device, and an interface to provide information to a user and/or receive information from a user.

This disclosure further comprises a method of controlling the function of a medical tool, in particular a medical device as described above. The method comprises determining the appearance of the indicator field.

Determining the appearance of the indicator field may comprise insertion of this information into the remote device over an interface, e.g., by a user that has visually determined the appearance of the indicator field. Determining the appearance of the indicator field may optionally comprise guiding a user which steps to take.

Determining the appearance of the indicator field may alternatively or additionally comprise taking a picture of the indicator field, and determining the appearance based on comparison with a reference image or reference area (reference field) in the indicator area. As a further alternative, determining the appearance of the indicator field may comprise electronically measuring the appearance of the indicator field, e.g., by measuring reflectance thereof, and comparing it to a calibrated value of the indicator field having its original appearance, optionally comprising determination of the difference between the (calibrated) value and the measured appearance.

Alternatively, determining the appearance of the indicator field may comprise electronically measuring the appearance of the indicator field, e.g., by measuring reflectance thereof, and comparing it to a measured value of a reference area (reference field), optionally comprising determination of the difference between the measured value of the reference area (reference field) and the measured appearance.

In particular, the indicator area including the indicator field may be arranged on a transparent part of the housing, in particular outside of the housing. Alternatively, the indicator area including the indicator field may be arranged on an, in particular opaque, housing, in particular inside of the housing. The reflectance of the indicator field may be measured between a light source, e.g., LED, and a sensor, e.g., photodiode, arranged inside the housing. The reflectance may in particular be measured through a transparent part of the housing or inside of the housing. Based on the determined appearance, the function of the medical tool is then controlled.

In particular, for example, if the appearance of the indicator field indicates that the device has been handled or stored at environmental parameters outside the safe parameter range, the device may be disabled. Alternatively, in particular if the appearance of the indicator field indicates that the device has been handled or stored outside of the environmental parameters recommended, but not been rendered unusable, but is now different, e.g., less sensitive, the (measurement) scale of the device may be adjusted.

This disclosure further comprises the use of a remote device as described above, the use in particular comprising use of the remote device in determining the appearance of the indicator field, e.g., by taking a picture thereof and optionally determining appearance of the indicator field, e.g., by comparison with a reference picture or a reference area in the indicator area. Alternatively, the remote device may comprise an interface to receive information on the determined appearance of the indication field, e.g., through a user interface. The use of the remote device may comprise forwarding this information, in direct or indirect form, to the controller of the medical device.

The use may alternatively or additionally comprise use of the remote device in displaying information about the medical device, in particular its function, to a user. It may in particular comprise displaying (to a user) that the device has been disabled, if the device has been disabled, or displaying a warning that the (measurement) scale has been adjusted, if the (measurement) scale has been adjusted. The remote control device may also be configured to display if the medical device works as intended.

This disclosure further comprises the use of a medical device, in particular a medical device as described above. The use thereof may, in particular, comprise using the medical device for determining the appearance of an indicator field, in particular measuring the reflectance thereof. The use of the medical device may further comprise controlling the function of the medical tool, e.g., disabling it or adjusting its (measurement) scale, in particular if the determined appearance of the indicator field indicates that the handling or storage has been outside of the safe environmental parameter conditions or changed due to environmental parameter conditions.

This disclosure further teaches the use of a control system for the function of a medical tool, in particular one as described before. The use may, in particular, comprise the steps described above for the use of a remote device and/or the medical device.

The method of controlling the function of a medical tool, the use of the control system and the remote device, as well as use of the medical device may additionally comprise steps of activating a medical device, in particular a medical device as described above. Activating the device may in particular comprise determining information allowing determination of the activation code. This may, e.g., comprise scanning the activation label in order to determine the activation code from the activation label, and may in particular comprise scanning the activation code on the activation label. The method may further comprise carrying out a verification step of the activation code, in particular comparing it with a reference activation code. The method further comprises sending an activation signal to the medical device based on the activation code, in particular if the verification step is successful. If the verification step is not successful, no activation signal may be sent to the medical device. The method steps may be carried out by the same entity, e.g., a remote device, or be carried out by different entities, e.g., the first by a remote device, the second by a remote server and the third again by the remote device. Other combinations are possible as well. The method may in particular comprise the steps described with regard to the medical device in European Patent Application titled “Medical Device comprising a temperature sensitive activation label” filed on June 9, 2023, with the European Patent Office as EP 23178 437.2.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows an exemplary medical device;

FIG. 2 shows an exemplary indicator area;

FIG. 3 shows a detail of an exemplary housing of a medical device;

FIG. 4 shows steps of a method of controlling the function of a medical tool; and

FIG. 5 shows an exemplary control system.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

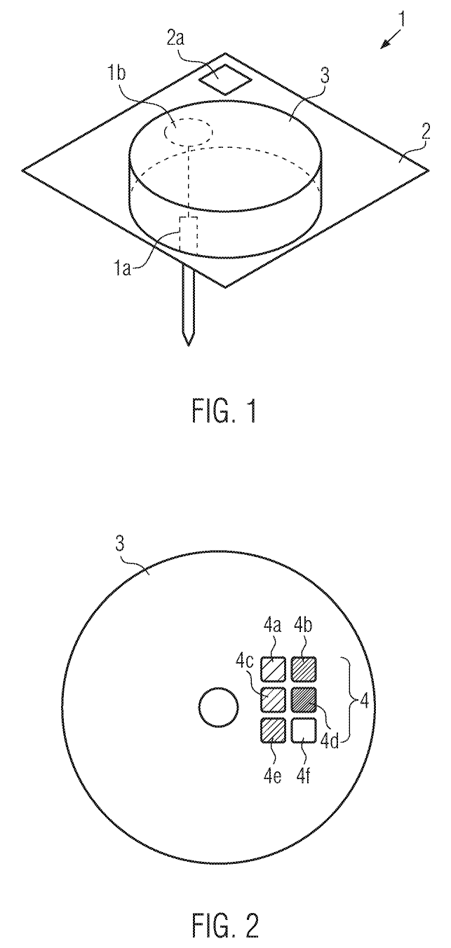

FIG. 1 shows an exemplary medical device 1 (as it would be schematically seen when applied to a user). The medical device 1 may be applied using an applicator (not shown). The exemplary medical device comprises a medical tool 1a and a controller 1b and a housing 3. In the exemplary medical device 1, which may in particular be a CGM system (continuous glucose monitoring system), the medical tool 1a, e.g., a glucose sensor allowing CGM, is connected to the controller 1b. This connection is schematically indicated by a line. The medical tool may in particular be a medical tool for subcutaneous insertion. The medical device 1 may further comprise a patch 2, which may be configured to attach the medical tool 1a and controller 1b to a user when the medical tool 1a is used. In particular, the medical tool 1a may extend through patch 2, thus allowing the tool to be, in particular subcutaneously, inserted into the skin of a user, while the part of the medical tool 1a extending on the other side of the patch may be connected to the controller 1b. Both the controller 1b and the part of the medical tool 1a on the side of the patch 2, which in use is not on the skin of a user, may be arranged in the housing 3. The medical device 1 may be delivered arranged in the applicator (not shown), and can be applied to the body of a user with the housing 3. After the patch 2 is applied, the applicator may be removed. The indicator area may in particular be arranged as coating, e.g., on a part of the housing or on a part of the patch (here exemplarily indicated by 2a). The medical device may be configured such that together with an activation signal or upon an activation signal, information about the determined appearance of the indicator field is needed for the controller to proceed with activation.

FIG. 2 shows an exemplary indicator area 4, as it may, for example, be applied or present on a housing 3 of a medical device, or on a patch or other part thereof (not shown), e.g., a medical device as shown in FIG. 1. The indicator area 4 comprises several fields 4a – 4f. In the particular example shown therein, one of the fields, e.g., field 4d may be an indicator field, while the other fields 4a, 4b, 4c, 4e, 4f may serve as reference fields, or more than one of the fields, e.g., fields 4b, 4d, 4f may be indicator fields, e.g., indicator fields for different environmental parameter conditions, whereas the other fields, e.g., 4a, 4c, 4e may be reference fields for the indicator field next to it. For example, indicator field 4b may irreversibly switch its appearance when a threshold humidity is reached or exceeded, indicator field 4d may irreversibly switch its appearance when a threshold humidity is reached or fallen below, and indicator field 4f may indicate when a further environmental parameter condition is reached, e.g., a certain humidity change is reached or exceeded.

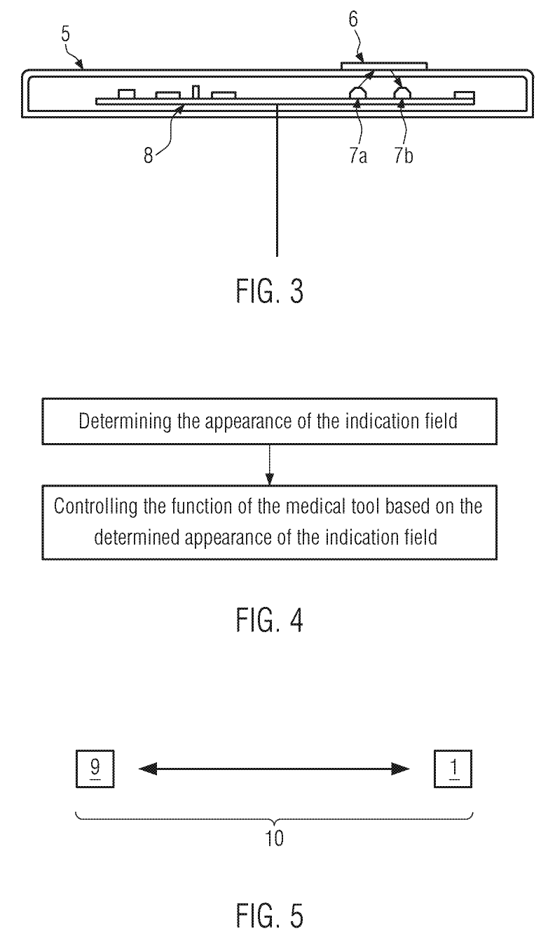

FIG. 3 shows a detail of an exemplary housing in cross section. In particular, the housing 5 comprises a printed circuit board assembly, which may comprise the controller or part of the controller or (at least in part) be comprised by the controller. An indicator field 6 is applied to the outside of the housing, in particular the part of the housing on the other side of the housing than the controller contained in the housing. The housing 5 is, at least in the part on which the indicator field 6 is applied, transparent. The indicator field may, in particular, be applied in the form of a coating, e.g., with ink sensitive to an environmental parameter.

The indicator area may comprise a single indicator field only (as shown in this example). In alternative examples (not shown), the indicator area may additionally comprise a reference area (e.g., comprising one or more reference field(s)), in particular if the indicator field is applied to the outside of the housing. In other examples (not shown), an indicator field, in particular a single indicator field (for each environmental parameter) only (without a reference field), may be arranged on the inside of a housing, e.g., on a part of the inner wall of the housing. In such examples, the housing may be opaque. For example, a single indicator field may be arranged on the side opposite of the housing’s wall to the indicator field 6 (not shown).

A light source 7, e.g., an LED, and a sensor 8, e.g., a photodiode, together may form or be part of a reflectometer comprised by the medical device.

The reflectometer is in this example connected to the controller. It can in particular be started and the results of it be read out by the controller. The controller may in particular be configured that before activation, or upon receipt of an activation signal, the reflectance of the indicator field 6 is measured to determine the appearance of the indicator field 6. The controller may in particular be configured to continue with the next planned steps based on the determined appearance, e.g., activation of the device, if the reflectance measured by the reflectometer corresponds to a calibration reflectance measured upon fabrication. The controller may be configured to disable the medical tool based upon the determined appearance, e.g., if the reflectance measured by the reflectometer deviates from the calibration reflectance more than predetermined value. In some examples, the controller may also be configured to change a start parameter, e.g., a (measurement) scale, in particular the sensitivity, of the medical tool, e.g., if the reflectance measured by the reflectometer deviates from the calibration reflectance more than a first and less than a second predetermined value.

FIG. 4 shows exemplary steps of a method of controlling the function of a medical device, which may also be used in a method of using the medical device or the control system. The exemplary method comprises the step of determining the appearance of the indicator field and controlling the function of the medical tool based on the determined appearance of the indicator field.

FIG. 5 shows an exemplary control system 10 for the function of a medical tool comprising a medical device 1, e.g., a medical device as shown in FIG. 1, and a remote device 9, e.g., a smartphone or tablet, in particular having an app for use of the medical device 1. The medical device 1 and the remote device 9 may, for example, be connected over a wireless or wired connection.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A medical device, comprising:
a medical tool;
an indicator area connected to the medical tool, the indicator area having an indicator field that irreversibly changes its appearance at a predetermined environmental parameter condition; and
a controller connected to the medical tool and configured to:
determine the appearance of the indicator field at the time of use of the medical tool, and
control the function of the medical tool based upon the determined appearance of the indicator field.

2. The medical device according to claim 1, wherein the medical tool comprises an insertion device and a housing.

3. The medical device according to claim 1, wherein the indicator area is arranged on the outside of a housing of the medical device.

4. The medical device according to claim 1, wherein the indicator area is arranged on a transparent part of a housing of the medical device.

5. The medical device according to claim 1, wherein the controller is configured to control the function of the medical tool by adjusting a start parameter of the medical tool and/or disabling the medical tool.

6. The medical device according to claim 1, wherein the medical device is configured such that at the time of use of the medical tool, the appearance of the indicator field is determined by a remote device.

7. The medical device according to claim 6, wherein the remote device comprises a portable communication device.

8. The medical device according to claim 1, wherein the appearance of the indicator field is determined by electronic measurement.

9. The medical device according to claim 1, further comprising a reflectometer configured to determine the appearance of the indicator field.

10. The medical device according to claim 1, wherein the medical tool comprises an insertion device and an opaque housing and wherein the indicator area is arranged inside of the housing.

11. The medical device according to claim 1, wherein a signal is sent to a remote device indicating the result of the control of the function of the medical tool.

12. The medical device according to claim 1, wherein:
the indictor field is sensitive to humidity or humidity changes;
the medical device further comprises an activation label comprising an activation code required for activating the medical device; and
the controller is configured to receive an activation signal from a remote device, and wherein the activation label comprises a temperature sensitive part that is configured to change its appearance at a predetermined temperature condition.

13. The medical device of claim 12, wherein the temperature sensitive part is configured to mask at least part of the activation code if the predetermined temperature condition is reached.

14. A control system for the function of a medical tool, comprising;
a medical device according to claim 1; and
a remote device.

15. A method of controlling the function of the medical device according to claim 1, the method comprising determining the appearance of the indicator field and controlling the function of the medical tool based thereon.

16. A method of using a remote device to control the function of the medical device according to claim 1, comprising using the remote device to determine the appearance of the indicator field and/or using the remote device to display information about the medical device.

17. The method according to claim 16, further comprising using the remote device to control the function of the medical tool.

18. The medical device according to claim 1, wherein the predetermined environmental parameter condition comprises a predetermined humidity condition, a predetermined humidity change condition, a predetermined temperature condition, or a predetermined temperature change condition.
